(19) United States
(12) Patent Application Publication
(10) Pub. No.: US 2016/0354522 A1
(43) Pub. Date: Dec. 8, 2016

(54) DRUG-ELUTING DEVICE FOR PROPHYLAXIS OR TREATMENT OF A DISEASE OR PATHOLOGY

(71) Applicant: FLUID BIOTECH INC., Calgary (CA)

(72) Inventors: ALIM P. MITHA, Calgary (CA); JOHN H. WONG, Calgary (CA); PALLAVI BHAMBRI, Edmonton (CA)

(21) Appl. No.: 15/149,006

(22) Filed: May 6, 2016

Related U.S. Application Data
(60) Provisional application No. 62/157,755, filed on May 6, 2015.

(51) Int. Cl. A61L 31/16; A61L 31/10; A61L 31/02; A61L 31/14

(52) U.S. Cl. CPC A61L 31/16; A61L 31/14; A61L 31/10; A61L 31/148; A61L 31/022; A61L 2300/604; A61L 2300/606; A61L 2300/608

(57) ABSTRACT

The invention disclosed herein generally relates to particular intravascular drug-eluting delivery devices and methods for manufacture and use in either the prophylaxis or treatment of a disease or pathology. In one aspect, the intravascular device may comprise a verapamil eluting stent with polymeric coating designed for the controlled release of a vasodilating drug in the prophylactic or remedial treatment of cerebral vasospasm.

### FIELD OF THE INVENTION

The invention disclosed herein generally relates to particular drug-eluting delivery devices and methods for manufacture and use in either the prophylaxis or treatment of a disease or pathology.

### BACKGROUND OF THE INVENTION

The in vivo delivery of therapeutic agents within the body of a patient can often be implemented using medical devices that may be temporarily or permanently placed at a target site within the body. Such medical devices can be maintained, as required, at their target sites for short or prolonged periods of time, delivering biologically active agents at the target site.

In accordance with certain delivery strategies, a therapeutic agent can be provided within or beneath a biostable or bioresorbable polymeric layer that is associated with a medical device. Once the medical device is placed at the desired location within a patient, the therapeutic agent can be released from the medical device with a profile that is dependent, for example, upon the nature of the therapeutic agent and of the polymeric layer, among other factors. An example of such a medical device includes a drug eluting stent.

The invention disclosed herein generally relates to particular drug-eluting delivery devices, methods for their manufacture and applications in either the prophylaxis or treatment of a disease or pathology.

One example of such an application is in the prophylactic or remedial treatment of cerebral vasospasm.

Cerebral circulation is supplied by the internal cartoid arteries and the vertebral arteries, whereas venous outflow is drained by the internal jugular veins and the vertebral veins. The subarachnoid space is the area between the arachnoid membrane and the pia mater surrounding the brain. The term “subarachnoid hemorrhage” refers to bleeding into the subarachnoid space. Hemorrhage may occur at the brain surface (extraparenchymal), for example from the rupture of congenital aneurysms at the circle of Willis, causing subarachnoid hemorrhage (SAH). SAH may occur spontaneously, usually from a cerebral aneurysm, or may result from trauma.

Those patients that survive SAH are also at risk of secondary complications. One major factor in the persistently high morbidity and mortality experienced after subarachnoid hemorrhage relates to the development of cerebral vasospasm. Cerebral vasospasm is a condition in which blood in the subarachnoid space causes contraction of the blood vessels and subsequent neurologic deficit or stroke. Cerebral vasospasm is a consequence of SAH, but also can occur after any condition that deposits blood in the subarachnoid space. Vasospasm occurs in up to 60% of subarachnoid hemorrhage patients and is the leading cause of death and disability after the initial hemorrhage. Cerebral vasospasm tends to occur on a delayed basis, usually after 4-21 days, with the peak incidence between 5-10 days after rupture.

The diagnosis of vasospasm is primarily clinical. Vasospasm can be asymptomatic. However, when the cerebral blood flow is below ischemic threshold, symptoms become apparent. Symptoms typically develop subacutely and may fluctuate. Symptoms can include excess sleepiness, lethargy, stupor, hemiparesis or hemiplegia, abulia, language disturbances, visual fields deficits, gaze impairment, and cranial nerve palsies.

The current mainstay of treatment for vasospasm can include hypertensive therapy, balloon angioplasty, and intra-arterial vasodilating drugs.

Hypertensive therapy can be fraught with a number of systemic complications resulting from the infusion of vasopressor medications and may require a patient to remain in an intensive care unit for cardiopulmonary monitoring.

Balloon angioplasty may typically be reserved for medically refractory cases, can have a short-term effect typically requiring multiple re-treatments, and can expose the patient to a significant risk of vessel perforation. Furthermore, balloon angioplasty may only be used to treat vasospasm in the large proximal vessels close to the Circle of Willis, without addressing the small distal vasculature. Balloon angioplasty can also be associated with a 10% risk of adverse effects such as displacement of aneurysm clips, stroke, vessel perforation and rupture, and has uncertain long-term histological effects on the treated arteries.

Intra-arterial vasodilating drugs can be effective at improving diameter in the proximal and distal blood vessels, by relaxing the tone of smooth muscle. However, intra-arterial vasodilating drugs typically provide short-term effects and often require retreatment.

It would be advantageous for there to be a drug delivery system comprising a drug-eluting device having a polymeric coating that allowed for the tailored release of one or more drugs or therapeutic agent.

It would also be advantageous to have a verapamil eluting prosthesis, such as a stent, designed for the controlled release of a vasodilating drug, in the prophylactic or remedial treatment of cerebral vasospasm.

### SUMMARY OF THE INVENTION

The invention disclosed herein generally relates to particular drug-eluting delivery devices, methods for manufacture and uses in either the prophylaxis or treatment of a disease or pathology.

In one embodiment, the invention comprises a drug eluting prosthesis for the controlled release of one or more drugs in the remedial or prophylactic treatment of a disease, the prosthesis comprising, a prosthesis body having an inner surface and an outer surface, at least one layer of biodegradable polymeric material bonded to at least one surface of the prosthesis body, the polymeric material being capable of absorbing and releasing the one or more drugs,at least one drug dispersed within at least one layer of the polymeric material, wherein the controlled release comprises control over initiation time for drug elution, control over duration time for drug elution, and control over quantity of drug being eluted.

In another embodiment the invention comprises a verapamil eluting stent for the controlled delivery of verapamil in the prophylactic or remedial treatment of cerebral vasospasm, the stent comprising, a stent body having a lumen with a diameter, an inner surface and an outer surface, at least one layer of biodegradable polymeric material bonded to at least one surface of the stent body, verapamil dispersed within at least one layer of the polymeric material, wherein the controlled delivery of verapamil comprises control over initiation time for verapamil elution, control over duration time for verapamil elution, and control over quantity of verapamil being eluted.

In another aspect the invention comprises the use of a drug eluting prosthesis for the controlled release of one or more drugs in the remedial or prophylactic treatment of a disease, the prosthesis comprising a prosthesis body having an inner surface and an outer surface, at least one layer of polymeric material bonded to at least one surface of the prosthesis body, at least one drug dispersed within at least one layer of the polymeric material, wherein the controlled release comprises control over initiation time for drug elution, control over duration time for drug elution, and control over quantity of drug being eluted.

In another aspect, the invention comprises the use of a verapamil eluting prosthesis for the controlled delivery of verapamil in the prophylactic or remedial treatment of cerebral vasospasm, the prosthesis comprising a prosthesis body having an inner surface and an outer surface, at least one layer of biodegradable polymeric material bonded to at least one surface of the prosthesis body, verapamil dispersed within at least one layer of the polymeric material; wherein the controlled delivery of verapamil comprises control over initiation time for verapamil elution, control over duration time for verapamil elution, and control over quantity of verapamil being eluted.

Additional aspects and advantages of the present invention will be apparent in view of the description, which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The subject matter which is regarded as the invention is particularly pointed out and distinctly claimed in the concluding portion of the specification. The invention, however, may best be understood by reference to the following detailed description of various embodiments and accompanying drawings in which:

FIG. 1 depicts a calibration curve of known verapamil concentrations plotted using UV spectroscopy at a wavelength of 278 nm;

FIG. 2 depicts a cumulative release profile of a plurality of stents and film;

FIG. 3 depicts a per day drug release profile of the plurality of stents and film of FIG. 2;

FIG. 4 depicts a scanning electron micrograph image showing the morphology of PLGA dip-coated stents by 10% w/v of PLGA in chloroform solution at 250×;

FIG. 5 depicts a scanning electron micrograph image showing the morphology of PLGA dip-coated stents by 10% w/v of PLGA in chloroform solution at 5000×;

FIG. 6 depicts a scanning electron micrograph image showing the morphology of PLGA dip-coated stents by 20% w/v of PLGA in chloroform solution at 500×;

FIG. 7 depicts a scanning electron micrograph showing the morphology of PLGA dip-coated stents by 20% w/v of PLGA in chloroform solution at 5000×;

FIG. 8 depicts a scanning electron micrograph showing the morphology of PLGA coated stents by 20% w/v of PLGA in chloroform solution using a spin coater at 250×;

FIG. 9 depicts a scanning electron micrograph showing the morphology of PLGA coated stents by 20% w/v of PLGA in chloroform solution using a spin coater at 1000×;

FIG. 10 depicts a scanning electron micrograph showing the morphology of PLGA coated stents by 15% w/v of PLGA in chloroform solution using an electrospinning technique at 100×;

FIG. 11 depicts a scanning electron micrograph showing the morphology of PLGA coated stents by 15% w/v of PLGA in chloroform solution using an electrospinning technique at 500×;

FIG. 12 depicts a scanning electron micrograph showing the morphology of PLGA coated stents by 20% w/v of PLGA in chloroform solution using an electrospinning technique at 500×; and

FIG. 13 depicts a scanning electron micrograph showing the morphology of PLGA coated stents by 20% w/v of PLGA in chloroform solution using an electrospinning technique at 2500×.

### DESCRIPTION OF THE PREFERRED EMBODIMENT

The present invention generally relates to drug eluting intravascular devices, as well as processes for preparation and uses of such devices. When describing the present invention, any term or expression not expressly defined herein shall have its commonly accepted definition understood by those skilled in the art. To the extent that the following description is of a specific embodiment or a particular use of the invention, it is intended to be illustrative only, and not limiting of the invention, which should be given the broadest interpretation consistent with the description as a whole.

### Intravascular Devices

An intravascular device of the present invention can be designed to elute one or more therapeutic agents for the prophylaxis or treatment of a disease or pathology in a target vascular region.

According to one embodiment, the intravascular device may be designed to elute a therapeutic agent at a controlled or predictable rate for an ascertainable period of time. The duration of the period can depend on the disorder that is being treated.

The term “intravascular device” as used herein can refer to a prosthesis that can be implanted within a bodily lumen or other body conduit, including a stent. A stent of the present invention can include stents which are covered or uncovered. The term “lumen”, as used herein, can refer to a cavity of a tubular organ such as a blood vessel.

The structure of the intravascular device may be formed of any suitable material including metals, ceramics, polymers, or any combination thereof. In alternative embodiments, the materials may be at least partially biodegradable. The at least partially biodegradable material preferably degrading in the body over time.

In one embodiment, the intravascular device of the present invention can be adapted for radial expansion in a bodily lumen or other body conduit, so as to allow fluid to flow through. A stent is an example of such a device.

In a particular embodiment, the intravascular device of the present invention may comprise a drug eluting vascular stent. The structure of a stent platform can comprise a scaffolding that includes a network of interconnecting structural elements such as strut or bar arms. The scaffolding may be formed, for example, from wires tubes or sheets of material rolled into a cylindrical shape. In one embodiment, the scaffolding design may be such that the stent may be radially compressed to allow for crimping and radial expansion once deployed within a bodily lumen or other body conduit at the treatment region.

The stent platform can be formed of any suitable biocompatible materials, including metals, ceramics, polymers, or any combination thereof. Such material should provide sufficient radial strength capable of withstanding structural loads imposed on the stent, as it supports the walls of a bodily lumen or body conduit. Suitable metals can include stainless steel, tantalum, gold, platinum-iridium alloy, molybdenum-rhenium alloy, nickel titanium alloy, cobalt alloys such as cobalt-chromium alloy, or any other malleable metals or resilient metals or alloys. Examples of suitable polymeric materials can include poly-L-lactic acid. In an embodiment, the stent platform may also be formed from a material which is at least partially biodegradable. Such materials can include any suitable polymeric materials, metallic materials, ceramic materials, or any combination thereof.

In one embodiment, the intravascular device of the present invention may comprise a covered stent. In an alternative embodiment, the intravascular device may comprise a stent which is uncovered.

In a particular embodiment, the intravascular device of the present invention can be a stent composed of a nickel titanium alloy (nitinol). In a further embodiment, the nitinol stent may also be self expandable, such as the Solitaire™ FR, available from eV3/Covidien (Irvine, Calif.).

### Polymer Coating

An intravascular device of the present invention may further include a polymer coating suitable for absorbing and releasing one or more therapeutic agents.

“Polymer” or “polymeric material” as used herein, can refer to a series of repeating monomeric units that have been cross-linked or polymerized. Any suitable polymer can be used to carry out the present invention. In some embodiments of the invention, only one polymer is used. In further embodiments, a combination of two or more polymers may be used. The polymers and the combinations of polymers can be used in varying ratios to provide coatings with differing properties.

In accordance with one embodiment, an intravascular device may include a single drug releasing base layer, that can include a polymeric material dispersed with one or more therapeutic agents applied to the surface of the device. The base layer may be above all or above substantially all, of the surface of the intravascular device and may function as a carrier or matrix for one or more of the therapeutic agents. The base layer may also provide a diffusion barrier or release controlling layer for the therapeutic agent. In alternate embodiments, the base layer may also provide a surface for further coating.

In accordance with an alternate embodiment, an intravascular device may comprise one or more additional upper layers built upon a base layer. An upper layer may include polymeric material and may or may not include additional therapeutic agents. An upper layer may be above all, or above substantially all of the surface of the intravascular device and base layer. An upper layer can function as a protective layer, allow for easier insertion or handling, act as a carrier or matrix for one or more therapeutic agents, and/or act as a rate controlling layer for the therapeutic agent incorporated in a layer below.

In an another embodiment, the intravascular device of the present invention may be bilayered, comprising a base layer and an upper layer. The upper layer of the device may comprise polymeric material while the base layer may comprise polymeric material dispersed or embedded with a therapeutic agent. While the upper layer may not include a therapeutic agent, it can function to create a delayed release effect of the therapeutic agent within the base layer.

In an alternative embodiment of the present invention, the intravascular device may be bilayered, wherein each of a base layer and upper layer may include a different therapeutic agent, in addition to polymeric material. Such an embodiment may allow a first therapeutic agent to initially be eluted from the outer layer and a second therapeutic agent to be subsequently eluted from the base layer.

In accordance with further embodiments, an intravascular device may be multi-layered, comprising more than two layers. As an example of one embodiment, the upper layers of an intravascular device may comprise an outer layer comprising a polymeric material with a first therapeutic agent and an inner or middle layer that may comprise polymeric material without a therapeutic agent. A base layer may comprise a polymeric material and a second therapeutic agent. Such device could provide elution of one drug with the outer layer, followed by a temporary delay through the non-drug eluting inner layer, and the re-elution of a therapeutic agent through the base layer.

Polymers useful in accordance with the present invention include, for example, stable polymers, biostable polymers, durable polymers, inert polymers, organic polymers, organic-inorganic copolymers, inorganic polymers, bioabsorbable, bioresorbable, resorbable, degradable, and biodegradable polymers. These categories of polymers may, in some cases, be synonymous, and is some cases may also and/or alternatively overlap.

In various embodiments, suitable polymers can include bioabsorbable and/or biodegradable polymers, including the following, combinations, copolymers and derivatives of the following: polylactides (PLA), polyglycolides (PGA), polycaprolactone (PCL), polylactide-co-glycolides (PLGA), polyanhydrides, polyorthoesters, poly(N-(2-hydroxypropyl) methacrylamide), poly(l-aspartamide), including the derivatives DLPLA—poly(dl-lactide); LPLA—poly(l-lactide); PDO—poly(dioxanone); PGA-TMC poly(glycolide-co-trimethylene carbonate); PGA-LPLA—poly(l-lactide-co-glycolide); PGA-DLPLA—poly(dl-lactide-co-glycolide); LPLA-DLPLA—poly(l-lactide-co-dl-lactide); and PDO-PGA-TMC—poly(glycolide-co-trimethylene carbonate-co-dioxanone), and any combination thereof.

As used herein, the term “bioabsorbable” or “biodegradable” refers to a polymer that is capable of being eroded or absorbed when exposed to bodily fluids such as blood and can be gradually reabsorbed, absorbed and/or eliminated by the body.

In a particular embodiment, the polymeric material can include poly(lactic-co-glycolic acid) (“PLGA”). PLGA is a copolymer of poly(lactic acid) (PLA) and poly(glycolic acid) (PGA). With respect to design and performance, PLGA may be considered a preferred biomaterial available for drug delivery.

PLGA can demonstrate desirable mechanical properties, biocompatibility, low toxicity, and biodegradability characteristics. For instance, the PLGA polymer can break down into lactic and glycolic acids, both of which can enter the tricarboxylic acid cycle and eventually be eliminated from the body as carbon dioxide and water. The potential for tuning degradation time by varying the monomer ratio during synthesis can also make PLGA a suitable biomaterial for use in conjunction with the intravascular devices of the present invention,

### Controlled Rate of Release

In an embodiment, an intravascular device of the present invention may be adapted to elute one or more therapeutic agents at a controlled rate for an ascertainable period of time. The duration of the period can depend on the disorder that is being treated.

The means for controlling the release of a therapeutic agent(s) can include the concentration of the therapeutic agent(s) selected and/or the polymeric material coating the device. The polymeric material may be capable of absorbing and releasing the therapeutic agent at a predictable or controlled rate when the device is implanted in a lumen or conduit of the body.

Typically, two major mechanisms regulate the release kinetics of a drug entrapped in a polymeric layer: 1) a diffusion-controlled mechanism in which the drug diffuses outwards through the bulk polymer due to concentration gradient, and 2) a degradation-controlled mechanism in which release of the drug depends on the hydrolytic degradation of the bulk polymer and erosion of polymer surface itself. The diffusion-controlled mechanism is likely dominant if drug release is faster than the expected time course of polymer biodegradation.

The composition of the polymeric material and/or the number of layers can provide a controlled drug delivery over a certain time frame, which may be dictated by the drug requirement and specific course of the disease.

In a further aspect, an intravascular device of the present invention may be adapted such that the initial release of the therapeutic agent can also be deferred to match the delayed clinical manifestations of a particular disease.

### Therapeutic Agent

An intravascular device of the present invention may be adapted for eluting one or more therapeutic agents.

The term “therapeutic agent” as used herein can refer to any of a variety of drugs or pharmaceutical compounds that can be used as active agents to prevent or treat a disease. The terms “therapeutic agent” and “drug” are used interchangeably.

The therapeutic agents may, if desired, also be used in the form of their pharmaceutically acceptable salts or derivatives (meaning salts which retain the biological effectiveness and properties of the compounds of this invention and which are not biologically or otherwise undesirable), and in the case of chiral active ingredients it is possible to employ both optically active isomers and racemates or mixtures of diastereoisomers. As well, a therapeutic agent may include a prodrug, a hydrate, an ester, a derivative or analogs of a compound or molecule.

It is also possible that the therapeutic agents of the present invention may also comprise two or more drugs or pharmaceutical compounds. Therapeutic agents include but are not limited to antibiotic agents, antiviral agents, analgesics, muscle relaxants, chemotherapeutic agents, intra-arterial vasodilating agents, calcium channel inhibitors, calcium channel antagonists, calcium channel blockers, transient receptor potential protein blockers, endothelin antagonists, or any combination thereof.

Examples of suitable therapeutic agents include but are not limited to: amlodipine, aranidipine, azelnidipine, bamidipine, benidipine, bepridil, cinaldipine, diltiazem, efonidipine, felodipine, gallopamil, isradipine, lacidipine, lamivudine (3TC), lemildipine, lercanidipine, milrinone, nicardipine, nifedipine, nilvadipine, nimodipine, nisoldipine, nitrendipine, manidipine, pranidipine, papaverine, temozolamide, vancomycin, verapamil, and the like, or any combination thereof.

In an embodiment, the therapeutic agent can comprise a chemotherapeutic agent such as temozolamide. In another embodiment, the therapeutic agent may comprise an antibiotic, such as vancomycin. In a further embodiment, the therapeutic agent may comprise a retroviral agent, such as lamivudine (3TC).

In a particular embodiment of the present invention, the therapeutic agent can comprise verapamil. Verapamil is an L-type calcium channel blocker of the phenylalkylamine class and a vasodilator that can be administered intra-arterially in patients with cerebral vasospasm.

### Applications for Use

The intravascular device of the present invention can be used to prevent or treat a disease, including any treatment of a disease in a mammal, such as preventing the disease, i.e. causing the clinical symptoms of the disease not to develop, inhibiting the disease (arresting the development of clinical symptoms), and/or relieving the disease (causing the regression of clinical symptoms).

Suitable applications can include, but are not limited to the prophylaxis or treatment of infections, cancer, cerebral vasospasm, etc.

In a particular aspect, the intravascular device of the present invention may be used for the treatment of cerebral vasospasm.

A potential complication of an aneurysmal subarachnoid hemorrhage (SAH) is the development of cerebral vasospasm. The term “cerebral vasospasm” refers to the narrowing of the large capacitance arteries at the base of the brain (i.e., cerebral arteries) following hemorrhage into the subarachnoid space, which may lead to reduced perfusion of distal brain regions, and subsequent neurologic deficit or stroke.

Vasospasm occurs in up to 60% of subarachnoid hemorrhage patients and can be the leading cause of death and disability after the initial hemorrhage. Cerebral vasospasm is a consequence of SAH, but also can occur after any condition that deposits blood in the subarachnoid space.

A general objective of vasospasm treatment can include increasing the diameter of the blood vessel and precluding or limiting the severity of arterial and symptomatic vasospasm. Treatment should be durable, and able to assuage the patient from symptoms over the time period typically associated with vasospasm with minimal short-term risks or long-term sequelae of the intervention.

Intra-arterial vasodilating agents that may be used as therapeutic agents for distending the blood vessels by relaxing the tone of smooth muscle, in accordance with the present invention, can include papaverine, nimodipine, nicardipine, milrinone, verapamil, and the like, each having their own mechanism of action

Verapamil is an L-type calcium channel blocker that may result in minimal to no elevation in ICP, and may be relatively safe. Consequently, in one embodiment of the present invention, verapamil may be selected as the therapeutic agent for use in the treatment of cerebral vasospasm.

### Verapamil-Eluting Prosthesis

In a particular embodiment, an intravascular device of the present invention can comprise a polymer coated prosthesis, such as stent, designed to elute verapamil over the time period typically associated with cerebral vasospasm. Cerebral vasospasm tends to occur on a delayed basis, usually after 4-21 days, with the peak incidence between 5-10 days after rupture of a subarachnoid hemorrhage.

The verapamil-coated stents of the present invention can be adapted to have different release behaviours congruent with concentration and/or layer composition. Varying the verapamil concentration as well as the number and composition of polymeric layers, can allow for controlled or targeted drug delivery over a certain time frame. Consequently, the verapamil concentration and whether a stent is bilayered or monolayered may be determined on the basis of the particular patient and the desired release profile.

An intravascular stent of the present invention may be monolayered, comprising a single base layer coating of PLGA and verapamil. In a particular embodiment, the intravascular stent can comprise a higher concentration of verapamil, for example, 30% by weight of verapamil to PLGA. In alternate embodiments, the intravascular stent may comprise even higher concentrations of verapamil, with an upper limit of approximately 50% by weight of verapamil to PLGA. In an alternate embodiment, a monolayered intravascular stent can comprise a lower concentration of verapamil, for example, 20% by weight of verapamil to PLGA. In alternate embodiments, the intravascular stent may comprise even lower concentrations of verapamil, with a lower limit of approximately 5% by weight of verapamil to PLGA. The monolayered stents may demonstrate a two-phase release profile, with an initial burst release followed by a slower rate of release.

In a further embodiment, the intravascular stent of the present invention may be bilayered, with a base layer comprising PLGA and verapamil and an upper layer comprising PLGA. In a particular embodiment, the bilayered intravascular stent can comprise a higher concentration of verapamil, for example, 30% by weight of verapamil to PLGA. In alternate embodiments, the intravascular stent may comprise even higher concentrations of verapamil, with an upper limit of approximately 50% by weight of verapamil to PLGA. In an alternate embodiment, a bilayered intravascular stent can comprise a lower concentration of verapamil, for example, 20% by weight of verapamil to PLGA. In alternate embodiments, the intravascular stent may comprise even lower concentrations of verapamil, with a lower limit of approximately 5% by weight of verapamil to PLGA. In instances where a delayed release of verapamil may be the most efficient manner of treatment, a bilayered intravascular stent may be appropriate.

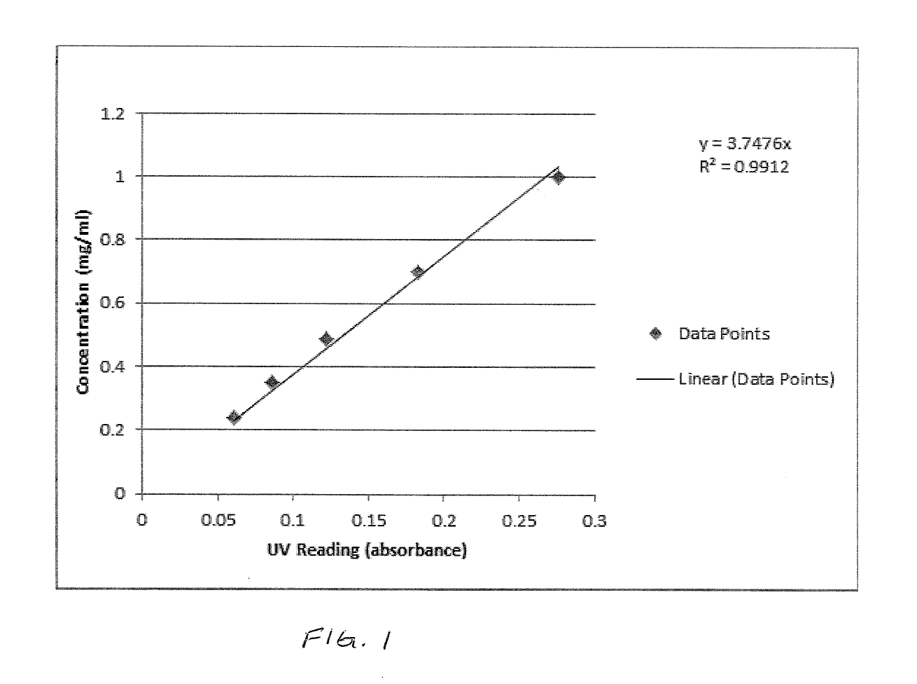

Referring now to FIG. 1, depicted therein is a calibration curve of known verapamil concentrations plotted using UV spectroscopy at a wavelength of 278 nm.

Samples of verapamil at known concentrations were prepared and then subjected to ultraviolet (UV) spectroscopy at a wavelength of 278 nm and a calibration curve was obtained. A linear curve was then plotted and the value of R² was determined. Concentrations and readings were taken until the value of R² was above 0.99.

The release kinetics of verapamil were examined in vitro by incubating individual verapamil-eluting stents or film in 10 mL of phosphate-buffered saline medium at 37° C. and pH 7.4. Aliquots (3 mL) of sampled medium were measured by UV spectroscopy at the same time each day (4-5 days each week) at a fixed wavelength of 278 nm. Results were expressed as cumulative drug release (sum of total milligrams of drug released up to and including the nth day).

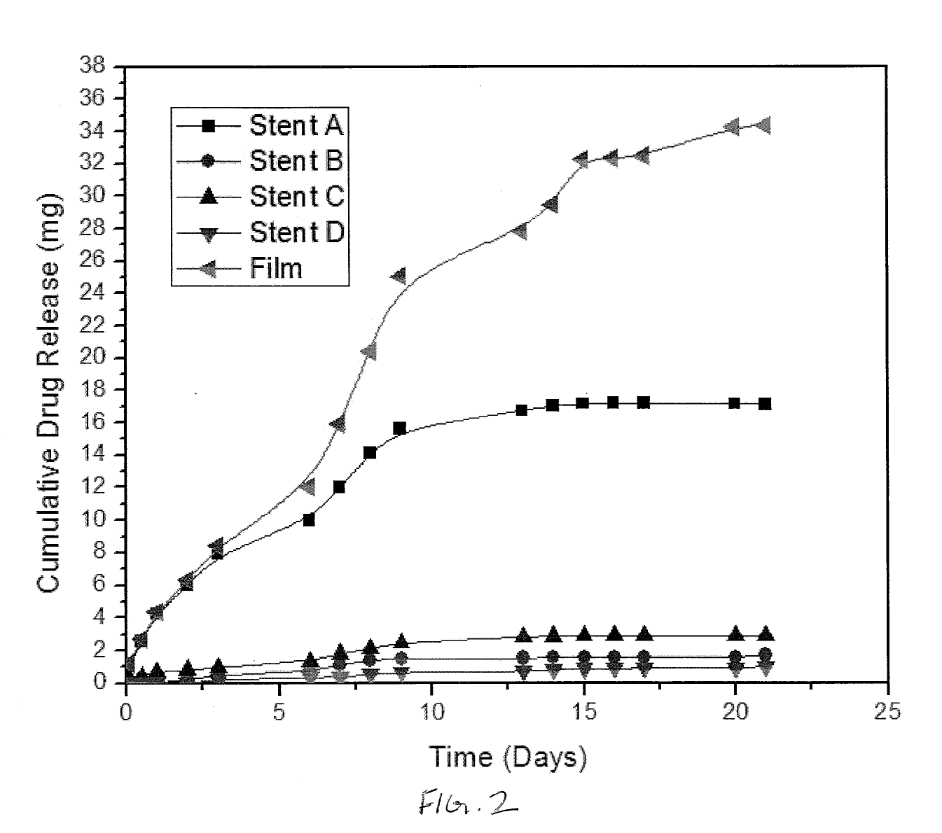

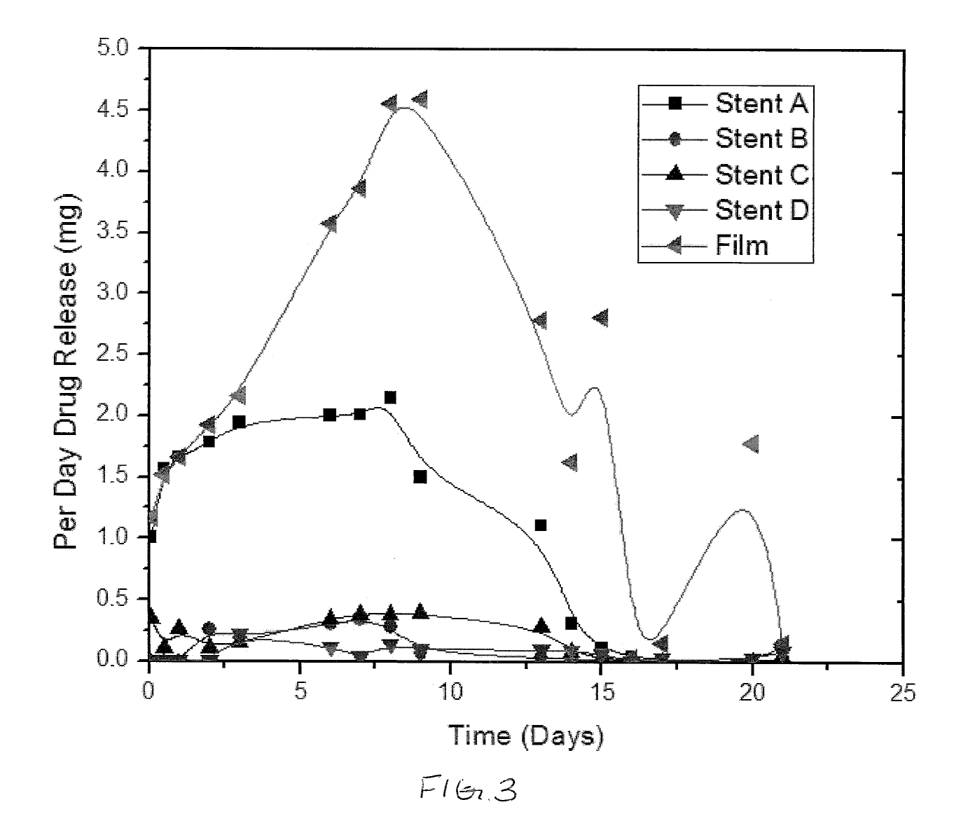

FIG. 2 depicts the cumulative release profile for particular monolayer and bilayer verapamil eluting stents and a verapamil-loaded film (i.e. a PLGA/verapamil layer without the stent scaffold) developed as a positive control. FIG. 3 depicts the per day drug release profile of the monolayer and bilayer verapamil eluting stents and the verapamil-loaded film.

“Cumulative drug release”, as defined herein, refers to the sum of total milligrams of drug released up to and including the nth day. “Per day drug release”, as defined herein, is the total drug released on nth day—total drug released on (n−1)th day.

Four different categories were developed (with three stents in each category). As depicted by FIGS. 2 and 3, Stent A comprises a monolayer stent having a higher concentration of verapamil (30% by weight of verapamil to PLGA) with a single layer coating of PLGA/verapamil. Stent B comprises a bilayer stent having a higher concentration of verapamil (30% by weight of verapamil to PLGA) with an upper layer of PLGA and a base layer of PLGA/verapamil. Stent C comprises a monolayer stent having a lower concentration of verapamil (20% by weight of verapamil to PLGA) with a single layer coating of PLGA/verapamil. Stent D comprises a bilayer stent having a lower concentration of verapamil (20% by weight of verapamil to PLGA) with an upper layer of PLGA and a base layer of PLGA/verapamil.

Stent A and Stent C (and the film) demonstrated a two-phase release profile characterized by a burst release phase followed by a sustained release phase. The initial burst release rate was similar between Stent A and Stent C. The persistent burst release phase of the film compared with Stent A can likely be attributed to the higher estimated total amount of drug used in the film.

The amount of drug loading in the polymer matrix can have an important role in the rate of drug release; matrices having higher drug content can possess a larger initial burst release compared with those having a lower content, which can be attributed to the polymer to drug ratio.

As depicted, the level of cumulative and daily verapamil release from monolayer Stent A was in significant contradistinction to that of monolayer Stent C, which had a lower concentration of verapamil. As such, this distinction may be attributed to the difference in verapamil concentration between the two stents.

The bilayer stents, Stent B and Stent D, demonstrated a delay in the initial onset of verapamil release of 48 hours and 72 hours, respectively.

As depicted, the addition of an upper layer of PLGA on top of a higher verapamil concentration/PLGA base layer in Stent B, caused a delay in the initial release phase. In comparison, a reduced verapamil concentration on the bilayer Stent D, resulted in a greater delay in the initial release phase, and a lower level of drug release.

Each of the bilayer stents, as well as the lower verapamil concentration monolayer Stent C, demonstrated a dampened release profile as compared to Stent A and the film. The dampening of the burst phase for the bilayer stents may be attributed to the upper layer of PLGA. The longer route of diffusion of verapamil due to the additional layer of coating may account for the slow release rate in the bilayer coated stents, resulting in an effect similar to reducing the concentration of verapamil, as in Stent C. Each of the stents demonstrated very reduced levels of verapamil release by 13 days.

As depicted in FIGS. 2 and 3, in the case of Stent A and the film, the rapid dissipation of verapamil present on the stent and film surface, as well as the hydrophilicity of verapamil, may have resulted in the initial burst release phase. The second release phase characterized by a slower and sustained release period may have been due to the degradation-controlled mechanism as opposed to diffusion of the drug.

Table 1 depicts the R² correlation coefficient between the experimental data and the Higuchi model obtained for the stents and the film.

The Higuchi model is a mathematical model used to quantify drug released from a matrix system. The Higuchi model is expressed by the equation:

Qₜ=A\sqrt{D(2C₀−Cₛ)Cₛt}

wherein Qt is the amount of drug released in time t per unit area A, C0 is the initial drug concentration, Cs is the drug solubility in the matrix media, and D is the diffusivity of the drug molecules (diffusion coefficient) in the matrix substance.

To further validate the drug release data obtained by the experiments, this data was compared to the results obtained through the Higuchi model.

After plotting the cumulative drug release versus square root of time, a linear R² value (correlation coefficient) was calculated. With regard to the monolayer stents, Stent A and Stent C, as well as the film, the plots were linear and showed a correlation coefficient R² close to unity. The bilayer stents, Stent B and Stent D, on the other hand, deviated from the Higuchi model. This deviation can likely be attributed to the additional outer layer of polymer coating.

TABLE 1

| Stent/Film | R² Correlation Coefficient |
|---|---|
| A | 0.9501 |
| B | 0.8991 |
| C | 0.9523 |
| D | 0.887 |
| Film | 0.9397 |

Drug-eluting stents, in particular, can be associated with delayed re-endothelialization which may result in increased thromboembolic risk in the cardiac circulation and may require longer-term treatment with antiplatelet medications. With regard to cardiac drug-eluting stents, its design allows the drug to be eluted over the course of several months, which may account for its persistent thromboembolic potential. The use of the verapamil-eluting stents of the present invention for cerebral vasospasm can, on the other hand, be designed to elute drug over a much shorter time period, perhaps resulting in reduced long-term risk. In addition, the PLGA stent coating as used for drug delivery in an embodiment of the present invention, may be less thrombogenic than bare metal.

The use of the drug-eluting stents of the present invention can also provide a method by which intraarterial vasodilators can be “infused” over time into the local circulation without requiring repeated treatments, while possibly avoiding potential systemic side effects of the agent.

In accordance with an aspect of the present invention, the drug concentration and polymer layers incorporated into a stent can be used to customize the level and onset of pharmacological action for the treatment of cerebral vasospasm depending on clinical severity and the desired timing of drug release, i.e. immediate or delayed. The desired timing of drug release may, for example, be immediate for patients who already have cerebral vasospasm. For patients who are at high risk of developing a vasospasm, the desired timing of drug release may be delayed, in which case the device may be used prophylactically.

A monolayered stent may, for example, be used for an immediate but slow release effect, whereas a bilayered stent may be preferable in instances in which a delayed timing may be desired.

The placement of an intravascular stent proximal to the affected circulation may provide a tailored treatment option for patients who are either symptomatic from severe vasospasm, or for those who are at particularly high risk of developing severe vasospasm. In either scenario, the drug may be needed only over the time course for cerebral vasospasm, which may typically not be beyond 21 days after a subarachnoid hemorrhage.

In one aspect for example of the present invention, a verapamil-eluting stent could be, placed in larger blood vessels proximal to the Willisian vessels, which would be void of critical branches.

### Drug-Eluting Intravascular Stents—Diseases or Pathologies

In the prophylaxis or treatment of other diseases or pathologies (tumor, infection, etc.) the desired timing of drug release may be tailored to the time course of the particular disease state being treated.

The desired timing of drug release may, for example, be immediate for patients who already have a disease/pathology downstream of the vascular territory in which the intravascular stent is placed. A stent may alternatively be used prophylactically in patients who are at high risk of developing a disease or pathology, in which case the desired timing of drug release may be delayed.

A monolayered stent may, for example, be used for an immediate but slow release effect of a therapeutic agent, whereas a bilayered stent may be preferable in instances in which the delayed release of the therapeutic agent is desired.

In the treatment of a tumor, for example, the therapeutic agents may elute over a time period shown to be effective or temporarily to minimize systemic side effects. An intravascular stent may be placed in a vascular territory proximal to the tumor, but which provides a blood supply to the tumor. In this way, the therapeutic agent can be targeted to the pathology while systemic side effects may be minimized as the agent may not be distributed to organs that do not involve the disease, as in the case of oral administration or intravenous administration of a therapeutic agent.

### Methods for Manufacture or Assembly

The surface morphology of a drug-eluting stent can play a role in clinical applications. Smooth surface coatings can diminish the endothelial damage to blood vessels, especially during delivery of the stent to the target site, while webbing between stent struts can impair expansion of the stent. Consequently, it is preferable that an intravascular stent have a smooth surface without webbings between stent struts.

The intravascular stents of the present invention may be coated with a bioabsorable polymer through any known coating technique in the art. Examples of suitable coating methods include, but are not limited to: dip coating, spin coating, electrospinning, spray coating, and the like.

### EXAMPLES

### Coating Morphology Experiments

Stents were first coated with PLGA polymer using three different coating techniques; dip coating, spin coating, and electrospinning. The morphology of the resulting stent coating was then analyzed with the help of a 30 kV scanning electron microscope (SEM) (FEI XL30). The stents for SEM observation were coated with gold prior to imaging.

Solitaire FR (ev3, Irivine, Calif.) nitinol stents were used. Polylactic acid-coglycolic acid 50:50, verapamil hydrochloride (purity>99%) and chloroform were obtained from Sigma Aldrich, Canada.

### a) Dip Coating

In the dip coating process, the target (stent) is immersed in the solution (PLGA chloroform in this example) then withdrawn, ideally at a constant speed. Excess solution is then drained from the surface and the solvent (chloroform) is allowed to evaporate. For the dip coating of stents, solutions of 10% weight/volume (w/v) and 20% w/v of PLGA in chloroform solvent were prepared by stirring overnight at room temperature. Stents were dipped inside the solutions for 5 minutes, then the solvent was evaporated by drying in a fume hood for 3 days. To ensure complete removal of chloroform, stents were placed under vacuum for a further 24 hours.

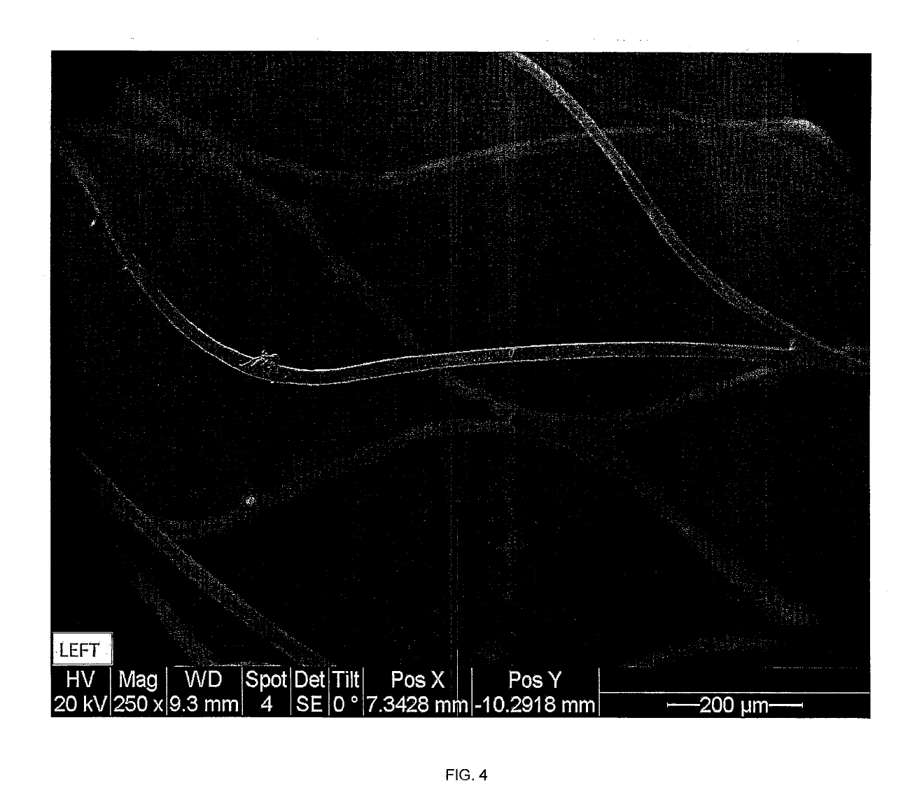

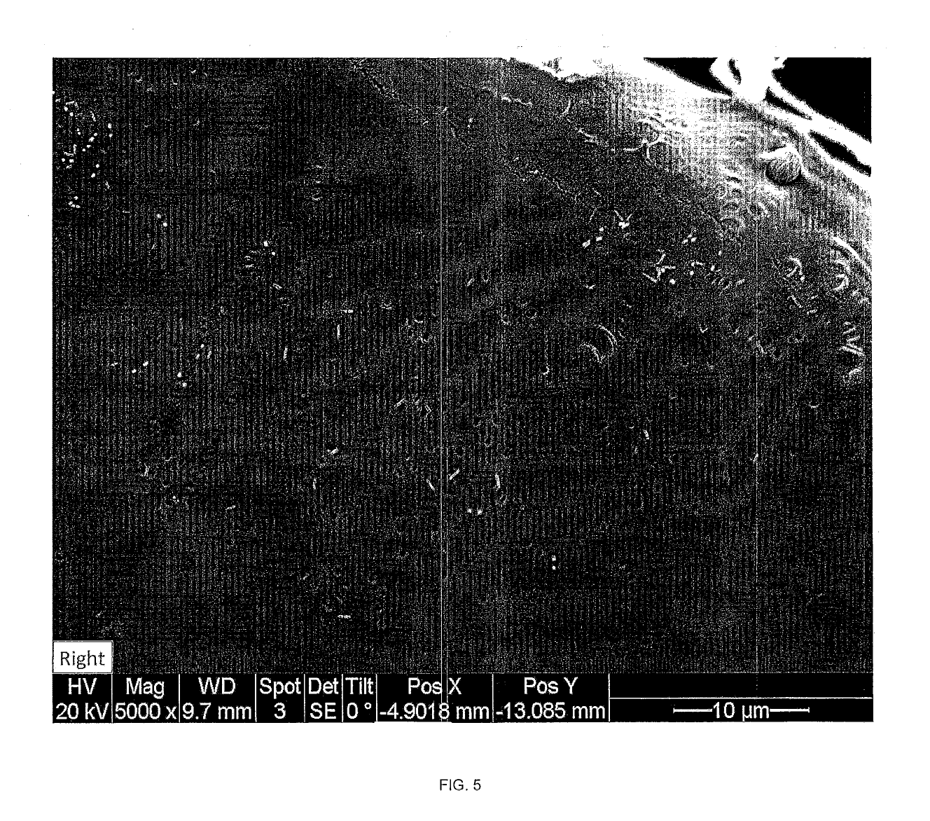

FIGS. 4-7 illustrate SEM images of dip coated stents by 10% w/v PLGA solution and 20% w/v solution, respectively.

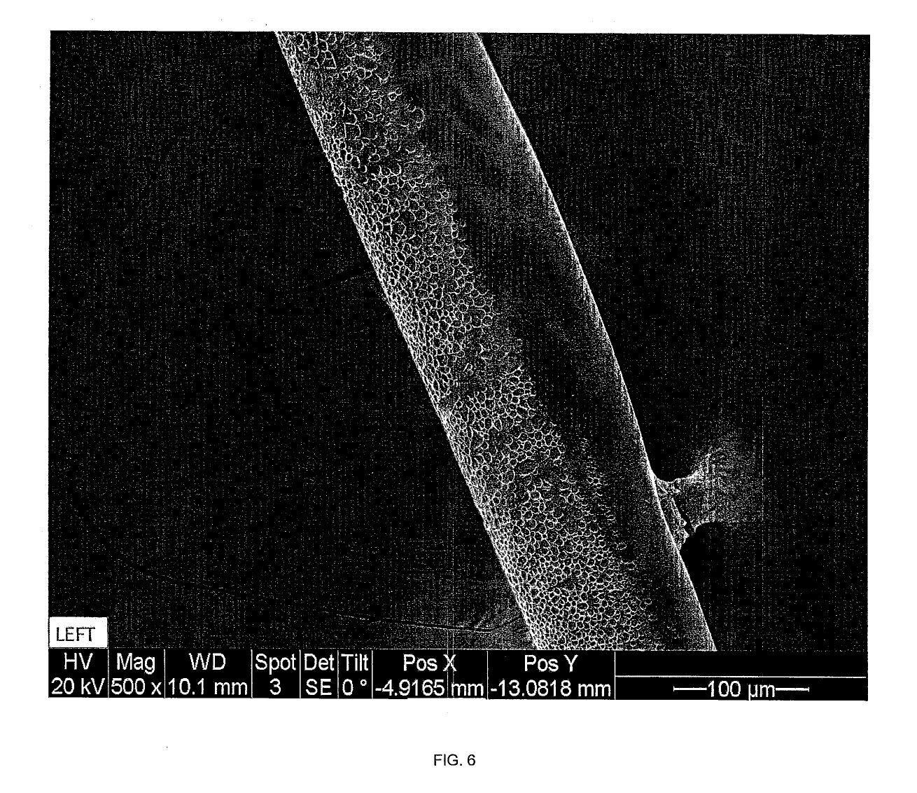

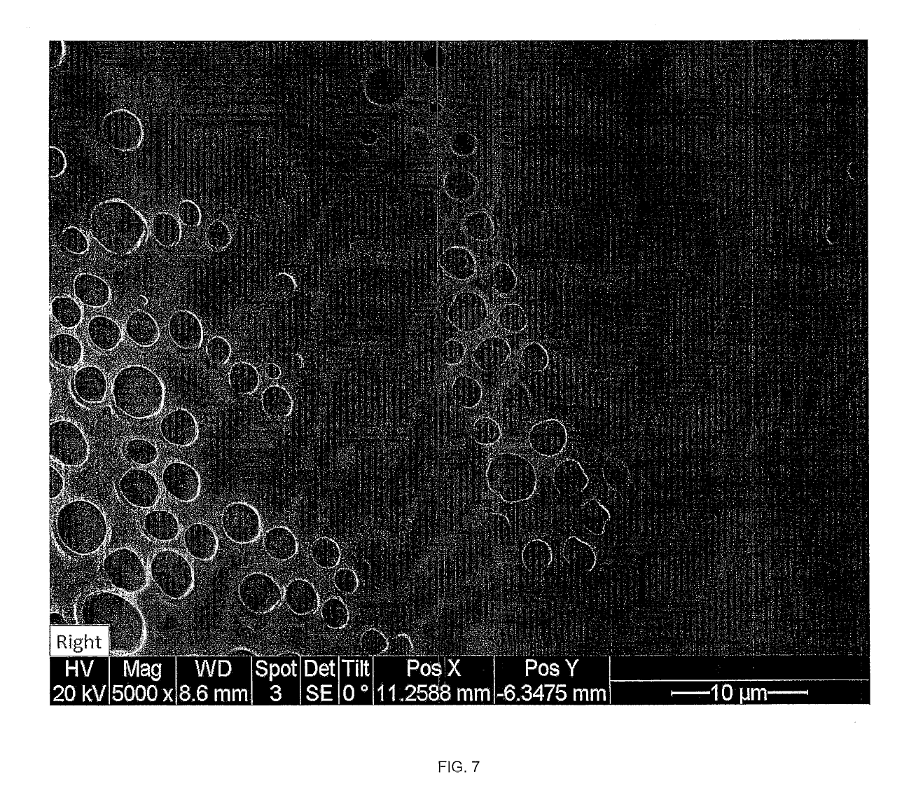

Both the 10% and 20% w/v PLGA concentrations resulted in a smooth coating on the stents. As depicted by FIGS. 6-7, bubbles were observed on the 20% w/v stent at high magnification, which may be attributed due to release of entrapped chloroform. Additionally, webbings between the 20% w/v stent struts could be seen with the naked eye (not depicted by the Figures) and likely occurred due to the high viscosity of the solution.

### b) Spin Coating

In the spin coating technique, a solution can be first applied to a target, then the target is rotated at high speeds to spread the solution by centrifugal force and to achieve a desired thickness. Most of the volatile solvent evaporates during the spinning process.

For the spin coating of stents, a 20% w/v solution of PLGA in chloroform solvent was prepared by stirring it overnight at room temperature. Stents were dipped inside the resulting solution for 5 minutes, then immediately mounted on the disc of a spin coater (Laurel) Technologies, North Wales, Pa.). The disc was rotated at 400 rpm for 10 minutes to slowly remove solvent and unattached polymer chains and in order to get a uniform coating on the stent surface. Coated stents were then kept in a vacuum for 24 hours to allow for complete evaporation of the solvent.

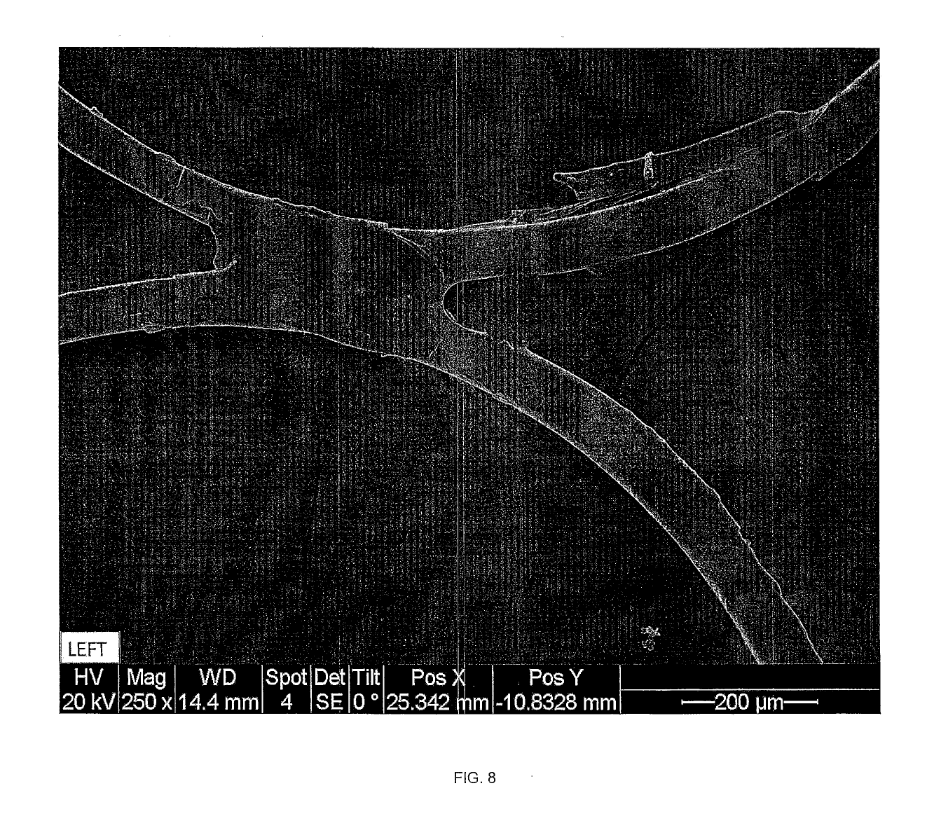

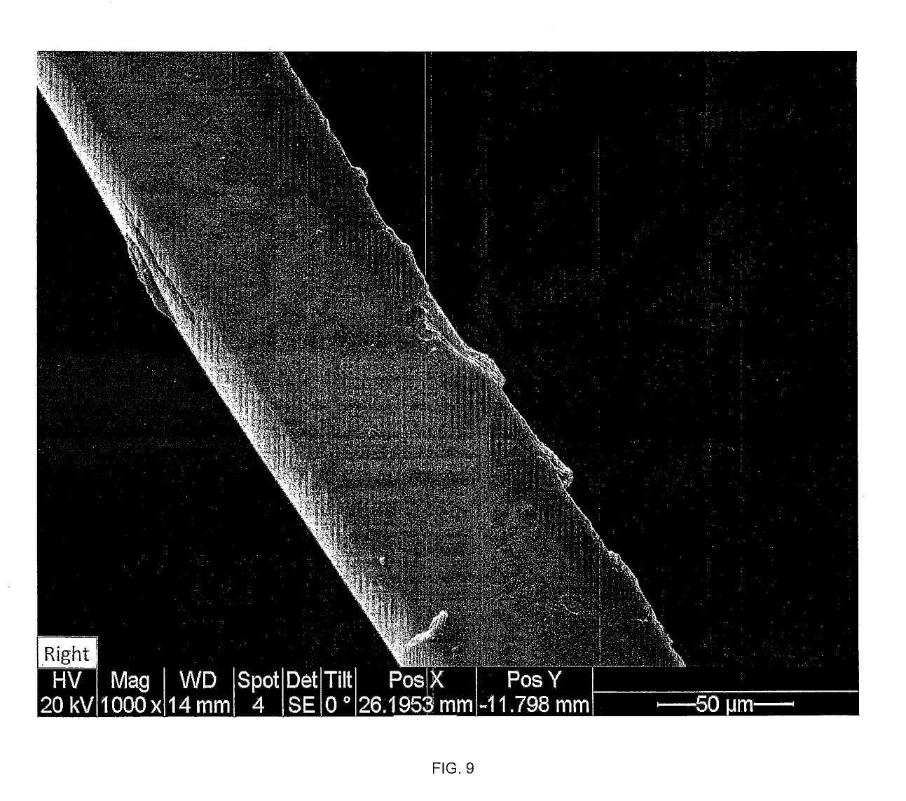

FIGS. 8-9 depict the SEM images of a stent coated by 20% w/v of PLGA chloroform solution using a spin coater.

Controlled release of chloroform using the spin coater at slow speeds facilitated a mostly uniform coating of PLGA to the stent surface, but with some minor surface irregularities. Using this method of coating, no webbings were found between the stent struts.

### c) Electrospinning

In the electrospinning technique, a capillary (filled with polymer solution) is connected to a high power supply; in the case of the PLGA solution, it accumulates electrostatic charges and when the electrical field is applied, the tip of the droplet outside the capillary is elongated. During the path between the capillary and the collector, the solvent evaporates, and nanofibers are produced by viscoelastic jet instabilities and deposited on the grounded collector. By changing the applied voltage and solution concentration, it is possible to adjust fiber diameters. By decreasing voltage or increasing solution concentration it is possible to obtain nano-scale droplets of PLGA solution deposited on surface of metallic stents where they merged and formed a smooth and uniform coating.

Solutions of 15% w/v and 20% w/v of PLGA in chloroform were utilized for electrospinning. The solution was electrospun on grounded stents using an applied voltage of 20 kV, a rotation speed of 700 rpm, and 5 cm work distance between capillary and stent. The stents were then kept in a vacuum for 24 hours to completely remove the solvent.

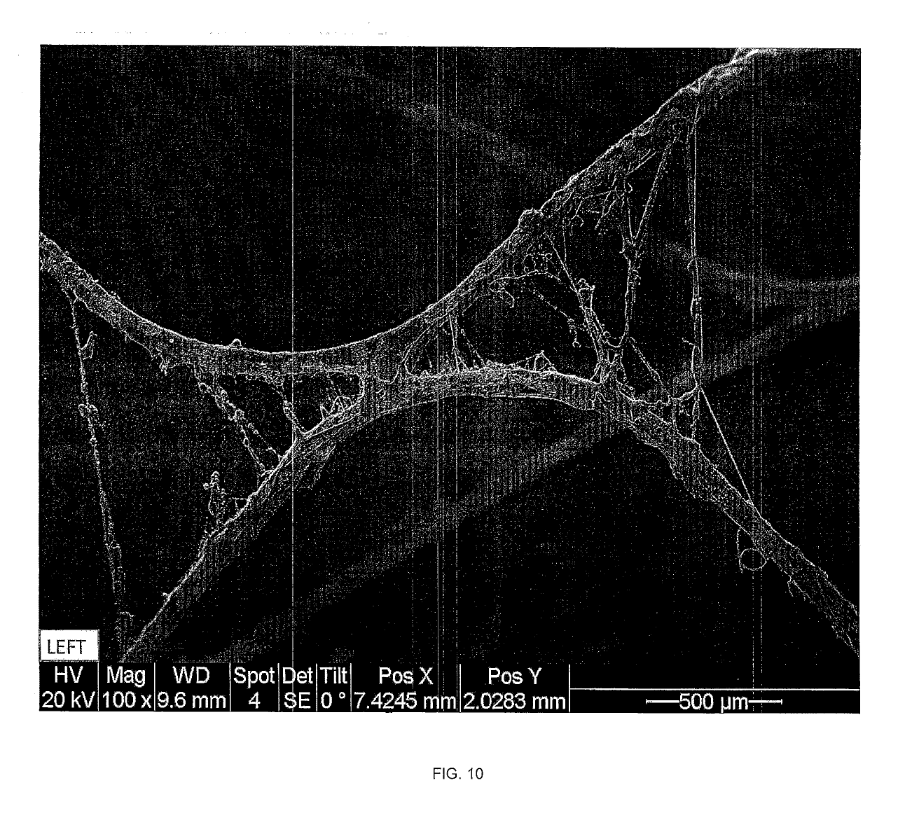

FIGS. 10-13 depict SEM images of stents coated using electrospinning technique with the help of 15% w/v and 20% w/v PLGA chloroform solution, respectively.

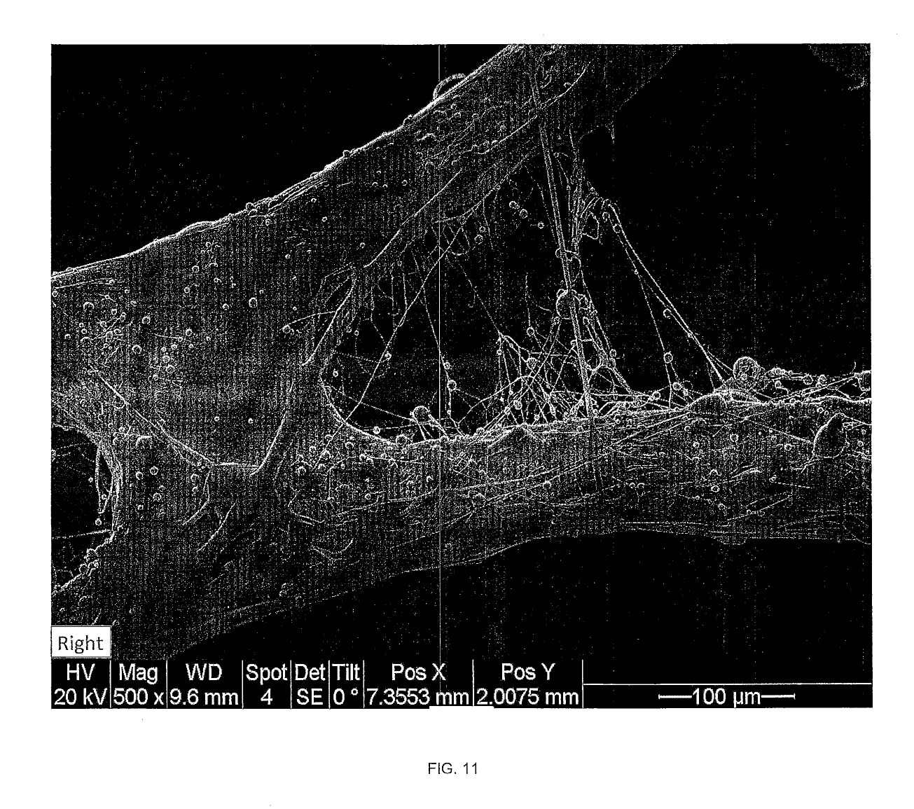

As depicted by FIGS. 10-11 specifically, some surface irregularities and web entanglements in the stent due to its low viscosity were observed with regard to the 15% solution.

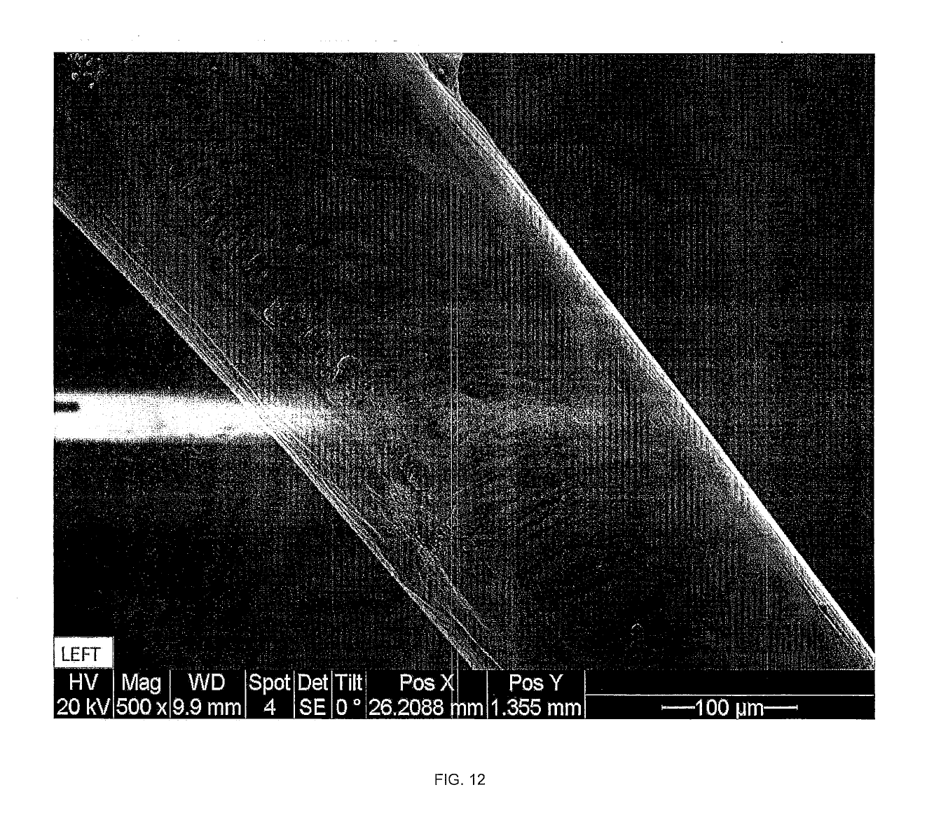

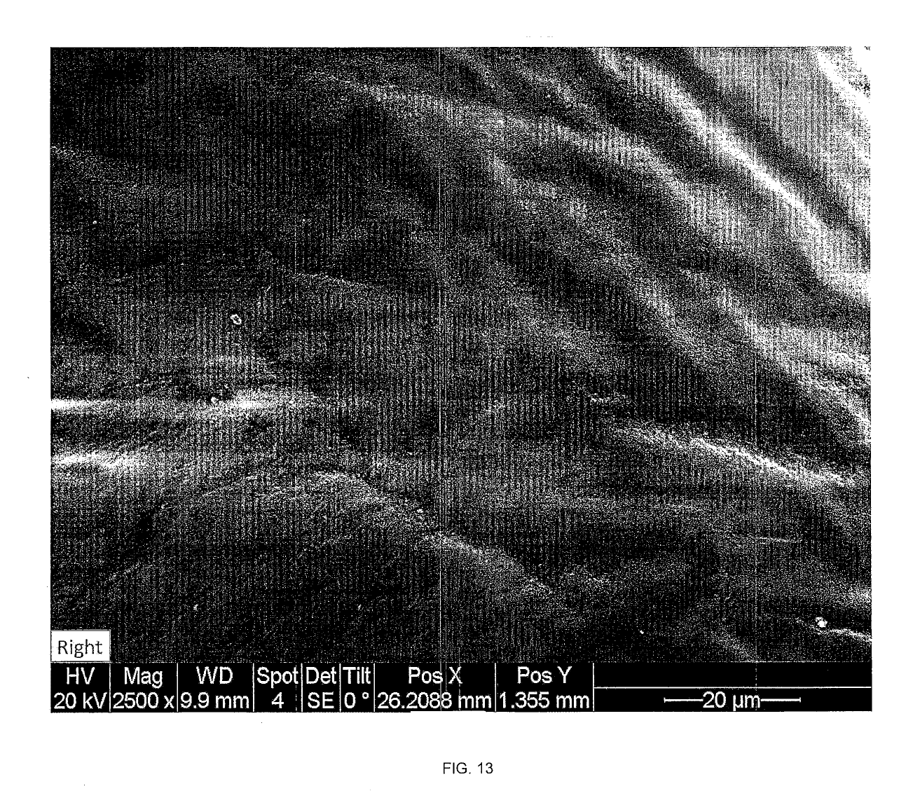

As depicted by FIGS. 12-13, a 20% solution prevented web formation due to formation of more droplets in nano-scale, as opposed to fibers, due to the higher viscosity of solution and resulted in a smooth and uniform coating. Droplet morphology also led to less web formation over stents. This differed from the dip coating method where a high viscosity solution resulted in increased web formation.

The study showed the feasibility to develop a verapamil-eluting stent using PLGA as the medium for drug elution.

As described, in a particular aspect, the stents may be coated through a dip coating method. However, as exhibited in the study, consideration to robust fiber formation between the stent struts may be required, which may impair expansion of stents during deployment.

In another aspect, a spin coating process may be used for coating an intravascular stent. As shown, spin coating provided a smooth coating similar to electrospinning and without fiber formation. However, as exhibited in the study, the spin coating process required 5 mL (20% w/v) while electrospinning required only 3 mL (20% w/v) of PLGA-chloroform solution.

In another aspect, the intravascular stents of the present invention may be coated through an electrospinning process. According to one embodiment, 20% w/v solution, for example may be used.

As demonstrated, the electrospinning process may yield a better balance between quality of coating and economy of the methods tested. As exhibited by the study, electrospinning required only 3 mL (20% w/v) of PLGA-chloroform solution. The electrospinning method may also provide ease of controlling the coating thickness of the PLGA layer. The coating thickness in this method linearly increases with processing time, which can allow for the development of stents which can release drug for a longer period of time. As such, the pharmacologic activity of stents coated using the electrospinning technique may be more easily modifiable. However, the present invention is not limited to the specific coating technique employed.

### Preparation of Verapamil-Eluting Stents

Stents were thoroughly cleaned with iso-propyl alcohol and 70% ethanol, then dried within a vacuum enclosure for 4 hours. Verapamil-eluting stents were prepared by dip coating.

15% w/v solution of PLGA chloroform solution was used for this purpose. Monolayer and bilayer stents (inner layer of PLGA/verapamil and outer layer of PLGA alone) were developed.

The bilayer stents were developed to match the initial release of the drug with the delayed clinical manifestations of the disease, soon after clip or coil occlusion of the culprit aneurysm in cases where severe vasospasm is anticipated to develop. Four stents were prepared:

Stent A: High concentration of verapamil (30% by weight of verapamil to PLGA) with a single layer coating of PLGA/verapamil.

Stent B: High concentration of verapamil (30% by weight of verapamil to PLGA) with inner layer of PLGA/verapamil and an outer layer of PLGA alone (bilayer coating).

Stent C: Low concentration of verapamil (20% by weight of verapamil to PLGA) with a single layer coating of PLGA/verapamil.

Stent D: Low concentration of verapamil (20% by weight of verapamil to PLGA) with inner layer of PLGA/verapamil and outer layer of PLGA alone (bilayer coating).

Drug-loaded PLGA films (i.e. a PLGA/verapamil layer without the stent scaffold) were developed as a positive control, as prior research on drug-eluting PLGA films had demonstrated a characteristic release profile of drug from these films.

Verapamil-loaded PLGA films were prepared by a solvent casting method using chloroform as the solvent. PLGA and verapamil (12% by weight of PLGA in solution) were dissolved in chloroform by stirring overnight to obtain 5% w/v solutions. The polymer solution (5 ml) was then poured into a glass petri dish and the solvent was slowly evaporated at room temperature for 4 days. Films were then placed under vacuum for 48 hours to remove any remaining solvent.

### In Vitro Release Test of Verapamil-Eluting Stents and Film

Samples of verapamil at known concentrations were prepared and then subjected to ultraviolet (UV) spectroscopy at a wavelength of 278 nm and a calibration curve was obtained. A linear curve was then plotted and the value of R² was determined. Concentrations and readings were taken until the value of R² was above 0.99.

Subsequently, the release kinetics of verapamil were examined in vitro by incubating the individual verapamil-eluting stents or film in 10 mL of phosphate-buffered saline medium at 37° C. and pH 7.4. Aliquots (3 mL) of sampled medium were measured by UV spectroscopy at the same time each day (4-5 days each week) at a fixed wavelength of 278 nm, then the aliquot was returned to the incubating solution. Results were expressed as cumulative drug release (sum of total milligrams of drug released up to and including the nth day).

Stent A and Stent C demonstrated a two-phase release profile characterized by a burst release phase followed by a sustained release phase. The initial burst release rate was similar between Stent A and Stent C and was identified by the high initial level of verapamil in the eluant on day 1. Their sustained release phases, however, diverged after day 5 due to lower drug concentration in Stent C. The persistent burst release phase of the film compared with Stent A and Stent C was likely due to the higher estimated total amount of drug used in the film. The bilayer stents (Stents B and D), a lower level of initial verapamil release into the eluent, followed by a slow sustained release phase. The dampening of the burst phase for the bilayer stents was likely due to the outer layer of pure PLGA which resulted in a slow initial release phase. Similarily, the additional layer of coating accounts for the the slow sustained release phase due to the longer route of diffusion of verapamil. The two bilayer coated stents and the stent with the single layer of low concentration PLGA/verapamil (Stent C) also showed a dampened release profile compared to Stent A and the film. All stents showed very reduced levels of verapamil release by 13 days.

In the case of Stent A and Stent C and the film, the rapid dissipation of verapamil present on the stent and film surface, as well as the hydrophilicity of verapamil, may have resulted in the initial burst release phase. The level of cumulative release from Stent A was in significant contradistinction to that of Stent C, which resulted simply from the difference in verapamil concentraton between the two stents. The addition of an outer layer of pure PLGA polymer on top of the inner layer of high concentration verapamil in PLGA (Stent B), caused a significant delay in the release of drug and yielding a release profile very similar to the bilayer stent with a lower concentration of verapamil (Stent D). This translates into the additional layer having a greater effect on the release profile compared to the overall concentration of drug, most likely because in these cases the diffusion controlled mechanism of drug release prevails. These results demonstrate that the drug concentration and polymer layers incorporated into the stent can be used to customize the level and onset of pharmacological action for the treatment of cerebral vasospasm depending on clinical severity and the desired timing of drug release, i.e. immediate or delayed. In all of the verapamil-eluting stents tested, drug release was sustained beginning by day 1 and was generally complete by day 13, which accounts for the period of peak incidence of vasospasm after SAH.

Observations of drug release kinetics were limited to the first 21 days which was intentional as the effects of cerebral vasospasm may not typically seen beyond this period. The study already demonstrated a plateau in verapamil release from all stents by about day 13.

In the preceding description, for purposes of explanation, numerous details are set forth in order to provide a thorough understanding of the embodiments of the invention. However, it will be apparent to one skilled in the art that these specific details are not required in order to practice the invention.

The above-described embodiments of the invention are intended to be examples only. Alterations, modifications and variations can be effected to the particular embodiments by those of skill in the art without departing from the scope of the invention.

What is claimed is:

1. A drug eluting prosthesis for the controlled release of one or more drugs in the remedial or prophylactic treatment of a disease, the prosthesis comprising:
a prosthesis body having an inner surface and an outer surface;
at least one layer of biodegradable polymeric material bonded to at least one surface of the prosthesis body, the polymeric material being capable of absorbing and releasing the one or more drugs;
at least one drug dispersed within at least one layer of the polymeric material;
wherein the controlled release comprises control over initiation time for drug elution, control over duration time for drug elution, and control over quantity of drug being eluted.

2. The prosthesis of claim 1, wherein the prosthesis is a drug eluting stent or scaffold having a lumen and capable of radial expansion, the prosthesis body formed from a material comprising metals, ceramics, polymers, or combinations thereof.

3. The prosthesis of claim 2, wherein the material forming the prosthesis body is at least partially biodegradable.

4. The prosthesis of claim 3, wherein the material comprises nitinol.

5. The prosthesis of claim 3, wherein at least a first drug is contained within a polymeric base layer bonded to at least one surface of the prosthesis body.

6. The prosthesis of claim 5, comprising a second polymeric layer that does not comprise a drug, the second polymeric layer built upon the base layer, and permitting the initiation of drug release from the base layer to be delayed.

7. The prosthesis of claim 5, comprising a second polymeric layer that comprises at least one drug, the second polymeric layer built upon the base layer, and permitting the initiation of drug release from the second polymeric layer prior to drug release from the base layer.

8. The prosthesis of claim 6, comprising a third polymeric layer that comprises at least one drug, the third polymeric layer built upon the second polymeric layer, the third polymeric layer permitting the initiation of drug release from the third layer prior to drug release from the base layer, the second polymeric layer permitting drug release from the base layer to be delayed.

9. The prosthesis of claim 1, wherein the polymeric material is selected from the group consisting of polylactides (PLA), polyglycolides (PGA), polycaprolactone (PCL), polylactide-co-glycolides (PLGA), polyanhydrides, polyorthoesters, poly(N-(2-hydroxypropyl) methacrylamide), poly(l-aspartamide), including the derivatives DLPLA—poly(dl-lactide); LPLA—poly(l-lactide); PDO—poly(dioxanone); PGA-TMC—poly(glycolide-co-trimethylene carbonate); PGA-LPLA—poly(l-lactide-co-glycolide); PGA-DLPLA—poly(dl-lactide-co-glycolide); LPLA-DLPLA—poly(l-lactide-co-dl-lactide), PDO-PGA-TMC-poly(glycolide-co-trimethylene carbonate-co-dioxanone), and copolymers, derivatives, and combinations thereof.

10. The prosthesis of claim 9, wherein the polymeric material comprises poly(lactic-co-glycolic acid) (“PLGA”).

11. The prosthesis of claim 1, wherein the at least one drug is selected from the group consisting of an antibiotic agent, antiviral agent, analgesic, muscle relaxant, chemotherapeutic agent, intra-arterial vasodilating agent, calcium channel inhibitor, calcium channel antagonist, calcium channel blocker, transient receptor potential protein blocker, endothelin antagonist, and combinations thereof.

12. The prosthesis of claim 11, wherein the at least one drug is selected from the group consisting of amlodipine, aranidipine, azelnidipine, bamidipine, benidipine, bepridil, cinaldipine, diltiazem, efonidipine, felodipine, gallopamil, isradipine, lacidipine, lamivudine (3TC), lemildipine, lercanidipine, milrinone, nicardipine, nifedipine, nilvadipine, nimodipine, nisoldipine, nitrendipine, manidipine, pranidipine, papaverine, temozolamide, vancomycin, verapamil, and combinations thereof.

13. The prosthesis of claim 12, wherein the at least one drug comprises verapamil.

14. A verapamil eluting stent for the controlled delivery of verapamil in the prophylactic or remedial treatment of cerebral vasospasm, the stent comprising:
a stent body having a lumen with a diameter, an inner surface and an outer surface. at least one layer of biodegradable polymeric material bonded to at least one surface of the stent body;
verapamil dispersed within at least one layer of the polymeric material;
wherein the controlled delivery of verapamil comprises control over initiation time for verapamil elution, control over duration time for verapamil elution, and control over quantity of verapamil being eluted.

15. The stent of claim 14 wherein at least one polymeric layer comprises polylactic-co-glycolic acid) (PLGA).

16. The stent of claim 15, comprising a base layer coating of PLGA and verapamil, verapamil being present at a concentration of between about 50% and about 5% by weight of verapamil to PLGA.

17. The stent of claim 15, wherein verapamil is present at a concentration of between about 35% and about 27% by weight of verapamil to PLGA, and wherein the controlled delivery of verapamil comprises an initial burst release phase followed by a sustained release phase.

18. The stent of claim 16, wherein verapamil is present at a concentration of between about 22% and about 15% by weight of verapamil to PLGA, and wherein the controlled release of verapamil by the stent comprises an initial burst release phase followed by dampened sustained release phase.

19. The stent of claim 16, comprising a second layer of PLGA that does not comprise a drug, the second PLGA layer built upon the base layer, and permitting the initiation of verapamil release to be delayed.

20. The stent of claim 19, wherein verapamil is present at a concentration of about 30% by weight of verapamil to PLGA, and the delay prior to initial onset of verapamil release is about 48 hours.

21. The stent of claim 19, wherein verapamil is present at a concentration of about 20% by weight of verapamil to PLGA, and the delay prior to the initial onset of verapamil release is about 72 hours.

22. Use of a drug eluting prosthesis for the controlled release of one or more drugs in the remedial or prophylactic treatment of a disease, the prosthesis comprising a prosthesis body having an inner surface and an outer surface, at least one layer of polymeric material bonded to at least one surface of the prosthesis body, at least one drug dispersed within at least one layer of the polymeric material, wherein the controlled release comprises control over initiation time for drug elution, control over duration time for drug elution, and control over quantity of drug being eluted.

23. Use of the prosthesis of claim 22, further comprising the steps of:
determining a typical drug requirement for the disease, determining a typical time course for the disease, and determining any typically delayed clinical manifestations for the disease;
implanting the prosthesis at a target location of a patient; and
contacting the prosthesis with one or more biological tissues or fluids in vivo to cause the at least one layer of polymeric material to degrade, controlling release of the drug into the patient;
wherein type and concentration of the drug corresponds to the typical drug requirement for the disease, and wherein drug release occurs substantially over the typical time course for the disease, and wherein any delay in the initial onset of drug release corresponds with any typical delayed clinical manifestations of the disease.

24. Use of the prosthesis of claim 23, wherein the disease is selected from the group consisting of cancer, infections or cerebral vasospasm.

25. The use of the drug eluting prosthesis of claim 23 wherein the at least one drug is selected from the group consisting of antibiotic agents, antiviral agents, analgesics, muscle relaxants, chemotherapeutic agents, intra-arterial vasodilating agents, calcium channel inhibitors, calcium channel antagonists, calcium channel blockers, transient receptor potential protein blockers, endothelin antagonists, and combinations thereof.

26. The use of the prosthesis of claim 23, wherein the at least one layer of polymeric material comprises a base layer comprising at least a first drug contained within the polymeric material, and a second polymeric layer that does not comprise a drug built upon the base layer, comprising:
contacting the second polymeric layer with one or more biological tissues or fluids in vivo to cause the second polymeric layer to degrade prior to degradation of the base layer, causing an initial delay in the release of the drug from the base layer;
contacting the base layer with one or more biological tissues or fluids in vivo to cause the base layer to degrade after substantial degradation of the second layer, thereby releasing the drug into the patient.

27. Use of a verapamil eluting prosthesis for the controlled delivery of verapamil in the prophylactic or remedial treatment of cerebral vasospasm, the prosthesis comprising a prosthesis body having an inner surface and an outer surface, at least one layer of biodegradable polymeric material bonded to at least one surface of the prosthesis body, verapamil dispersed within at least one layer of the polymeric material; wherein the controlled delivery of verapamil comprises control over initiation time for verapamil elution, control over duration time for verapamil elution, and control over quantity of verapamil being eluted.

28. Use of the prosthesis of claim 27, wherein the prosthesis has a release profile comprising an immediate burst release and subsequent sustained release, an immediate sustained release, or a sustained release after a delay in the initial onset of release.

29. Use of the prosthesis of claim 28, further comprising selecting a desired release profile for the verapamil, the release profiles comprising an immediate burst release and subsequent sustained release, an immediate sustained release, or a sustained release after a delay in the initial onset of release;
placing the prosthesis proximal to blood vessels in spasm, the prosthesis release profile corresponding with the desired release profile for the verapamil;
contacting the at least one layer of polymeric material with one or more biological tissues or fluids in vivo to cause degradation of the polymeric material, thereby releasing verapamil into the patient in accordance with the release profile.

30. Use of the prosthesis of claim 29, wherein the release of verapamil comprises a time period of up to about twenty-one days from the initial onset of verapamil release.

31. Use of the prosthesis of claim 29, wherein the release of verapamil substantially plateaus after about thirteen days from the initial onset of verapamil release.

32. Use of the prosthesis of claim 29, wherein the at least one polymeric layer comprises a base layer coating of PLGA and verapamil, wherein the verapamil present at a concentration of between about 35% and about 27% by weight of verapamil to PLGA, and wherein the delivery of verapamil comprises an initial burst release phase followed by a sustained release phase.

33. Use of the prosthesis of claim 29, wherein the at least one polymeric layer comprises a base layer coating of PLGA and verapamil, wherein the verapamil is present at a concentration of between about 22% and about 15% by weight of verapamil to PLGA, and wherein the delivery of verapamil comprises an initial burst release phase and followed by an dampened sustained release phase.

34. The use of the verapamil eluting prosthesis of claim 29 wherein the at least one polymeric layer comprises a base layer coating of PLGA and verapamil present at a concentration of about 30% by weight of verapamil to PLGA, and a second layer of PLGA that does not comprise a drug, the second PLGA layer built upon the base layer, wherein the delay prior to initial onset of verapamil release is about 48 hours.

35. The use of the verapamil eluting prosthesis of claim 29 wherein the at least one polymeric layer comprises a base layer coating of PLGA and verapamil present at a concentration of about 20% by weight of verapamil to PLGA, and a second layer of PLGA that does not comprise a drug, the second PLGA layer built upon the base layer, wherein the delay prior to initial onset of verapamil release is about 72 hours.

36. A method for manufacturing the drug eluting prosthesis of claim 1, comprising
preparing a solution comprising a polymeric material, a drug and a solvent;
coating at least a portion of a surface of the prosthesis body, wherein the coating method is selected from the group consisting of dip coating, spin coating, electrospinning, and spray coating.

37. The method of claim 36, wherein the coating method comprises electrospinning.
